(19) United States
(12) Patent Application Publication
(10) Pub. No.: US 2002/0150915 A1
(43) Pub. Date: Oct. 17, 2002

(54) Promoter sequences

(76) Inventors: Anders Berkenstam, Stockholm (SE); Goran Bertilsson, Stockholm (SE); Patrik Blomquist, Lidingo (SE)

Correspondence Address: JANIS K. FRASER, Ph.D., Fish & Richardson P.C., 225 Franklin Street, Boston, MA, 02110-2804, US

(21) Appl. No.: 09/957,997

(22) Filed: Sep. 21, 2001

Related U.S. Application Data
(60) Provisional application No. 60/238,895, filed on Oct. 10, 2000.

(30) Foreign Application Priority Data
Sep. 22, 2000 (SE) 0003393-6

(51) Int. Cl. C12Q 1/68; C07H 21/04; C12N 9/02; C12P 21/02; C12N 5/06

(52) U.S. Cl. 435/6; 435/189; 435/325; 435/320.1; 435/69.1; 536/23.2

(57) ABSTRACT

The present invention relates to an isolated human Cytochrome P-450 3A7 (CYP3A7) promoter region. The invention also relates to screening methods for agents modulating the expression of CYP3A7, such agents being potentially useful in modulating metabolism of endogenous and/or exogenous compounds, drug interaction, toxicity and/or bioavailability of drugs.

### CROSS REFERENCE TO RELATED APPLICATIONS

This application claims priority from Swedish Patent Application No. 0003393-6, filed Sep. 22, 2000, and U.S. Provisional Patent Application Serial No. 60/238,895, filed Oct. 10, 2000. These applications are incorporated herein by reference in their entirety.

### TECHNICAL FIELD

The present invention relates an isolated human Cytochrome P-450 3A7 (CYP3A7) promoter region. The invention also relates to screening methods for agents modulating the expression of CYP3A7, such agents being potentially useful in modulating metabolism of endogenous and/or exogenous compounds, drug interaction, toxicity and/or bioavailability of drugs.

### BACKGROUND

The cytochrome P-450 hemoproteins (P-450) are important metabolizers of both endogenous and exogenous compounds (Nelson et al., 1996). The human P-450 3A superfamily contains three functional members: CYP3A4, CYP3A5 and CYP3A7. The CYP3A4 isoform is the major metabolizer of therapeutic drugs in adults and plays a central role in the steroid hormone metabolism. More than 60% of therapeutic drugs are metabolized by CYP3A4 (Li et al., 1995). While CYP3A4 is the predominant P-450 in adult liver, the CYP3A7 isoform (SEQ ID NOS: 2 and 3) is the major P-450 in fetal liver (Yang et al., 1994). CYP3A7 is also expressed in placenta and endometrium, and in low amounts in adult liver, lung, kidney and tumors (Hakkola et al., 1996; Kivisto et al., 1996; Kolars et al., 1994; Murray et al., 1999; Schuetz et al., 1993). CYP3A7 has been postulated to protect the growing embryo against xenobiotics and steroids that passes the placenta (Schuetz et al., 1993). There are however several procarcinogenes that are converted into their active forms by CYP3A7 or CYP3A4 (Shimada et al., 1996). Consequently, an altered expression of CYP3A7 or CYP3A4 could cause problems by changing steroid homeostasis, by activating substances to make them toxic, or by inactivating drugs.

The CYP3A4 and CYP3A7 isoforms are 95% identical on the nucleotide level, indicating a close evolutionary relationship. The substrate specificities for the two isoforms are also similar (Hakkola et al., 1998). Since both CYP3A4 and CYP3A7 expression is controlled by several drugs and hormones, a better understanding of the transcription factor(s) involved in the regulation of the corresponding genes is of great importance in avoiding problems due to drug interaction. The xenobiotics that activate the genes, and their homologues, differ between species (Kocarek et al., 1995). This could be explained by the species difference in the activation profile of the transcription factor that activates CYP3A genes in response to xenobiotics. However, it may be difficult to predict effects on drug metabolism based on experiments done in other species.

The nuclear receptor designated Pregnane Activated Receptor, PAR (NR1I2, also called PXR and SXR), has been cloned and found to be activated by several xenobiotics that influence CYP3A4 or CYP3A7 expression (Bertilsson et al., 1998; Blumberg et al., 1998; Kliewer et al., 1998; Lehmann et al., 1998; see also WO 99/19354; WO 99/31129; WO 99/35246; and WO 99/48915). Since PAR response elements (PREs) were found in the proximal CYP3A4 and CYP3A7 promoters and were shown to be responsive to PAR, it has been suggested that PAR is a xenobiotic sensor (Pascussi et al., 1999). It has also been shown that the PAR activation profile resembles the profile of CYP3A gene induction in different species (Barwick et al., 1996; Jones et al., 2000). This has been further supported by transgenic experiments where mice lacking endogenous PAR expressed the human PAR in the liver. The activation of the murine CYP3A11 gene (the CYP3A4 homologue) by xenobiotics was “humanized” in such mice (Xie et al., 2000). The fact that the activation profile of the CYP3A4 and CYP3A7 genes correlates with the species origin of PAR suggests that PAR is the key xenobiotic sensor in CYP3A4 and presumably in CYP3A7 regulation.

In addition to the PREs identified in the proximal promoter sequences of CYP3A4 and CYP3A7, a xenobiotic response element (XREM) has been identified in the distal part (7.8 to 7.6 kb upstream) of the CYP3A4 promoter (Goodwin et al., 1999). The XREM contained two PREs and were, in collaboration with the proximal PRE, shown to mediate the transcriptional response to rifampicin in the presence of transiently expressed PAR.

Some environmental agents have been shown to disrupt the endocrine functions in many species through a variety of pathways including the change of steroidogenesis. Nonylphenol and phtalic acid, two of these endocrine-disrupting chemicals (EDCs), have been demonstrated to induce PXR-mediated CYP3A expression (Masuyama et al., 2000).

The PAR receptor has a similar DNA binding preference as the Constitutive Androstane Receptor (CAR) (Sueyoshi et al., 1999). CAR and PAR share some xenobiotic and steroid ligands (Moore et al., 2000). CAR has also been shown to activate the CYP2B gene via a phenobarbital responsive element. Recent experiments have shown that although CAR can activate a CYP3A4 XREM reporter, PAR appears to be the dominant transcription factor (Goodwin et al. 1999).

Only the proximal part (1012 bp) of the CYP3A7 promoter sequence has previously been identified (GenBank™ Accession No. AF181861; SEQ ID NO: 4) and consequently it has not been known whether the distal part of the CYP3A7 promoter contains response elements for transcription factors that could regulate the transcription of CYP3A7.

### BRIEF DESCRIPTION OF THE DRAWINGS

FIG. 1: (A) Schematic illustration of the CYP3A7 promoter. Restriction enzyme sites relevant for the cloning of different reporter construct have been indicated. (B) Comparison of the CYP3A7 and CYP3A4 promoters. Regions with high sequence identity (90%) are indicated by an open bar and regions with low identity (˜25%). XREM, dNR3 and pNR are shown by black boxes. (C) Sequence comparison of the different PREs from CYP3A7 and CYP3A4. Nucleotides that differ are underlined. The different half-sites of the PREs are indicated by bold letters and by arrows.

FIG. 2: Identification of the XREM as a PAR responsive element in the CYP3A7 promoter. Schematic drawings of the CYP3A7-luciferase reporter constructs are shown to the left, with the XREM, dNR3 and pNR indicated by black boxes. The graph shows the fold induction of cells treated with 10 μM rifampicin as compared to vehicle (DMSO). The black columns symbolize cells transfected with a plasmid expressing hPAR-2 and the white columns symbolizes cells transfected with an empty expression plasmid. Data are mean +/−SD for a typical experiment with six values in each group. The experiment has been repeated on five different occasions with similar result.

FIG. 3: Identification of CAR as transcription factor for CYP3A7. C3A cells were transfected with Cyp3A7 promoter with (Cyp3A7-9302) or without (CYP3A7-7478) the XREM and with CAR expression vector (CAR) or with empty (pcDNA3) expression vector. Cells were treated with vehicle (DMSO) or 10 μM of rifampicin or clotrimasol. Relative luciferase values represent luciferase activity compensated for the alkaline phosphatase reporter. The data are mean values +/−SD for six wells of a typical experiment

### DISCLOSURE OF THE INVENTION

This invention relates to the cloning and sequencing of the upstream promoter sequence (SEQ ID NO: 1) of the CYP3A7 gene. The CYP3A7 promoter sequence shows more than 90% identity to the CYP3A4 promoter up to approximately 8.8 kb upstream. Transient transfection experiments using a reporter plasmid containing the CYP3A7 promoter identified a region containing a XREM-like sequence (positions 3330 to 3551 in SEQ ID NO: 1) as the region containing the PAR response element. We conclude that CYP3A7 is regulated by PAR and that this regulation is mainly mediated via the CYP3A7 XREM. Experiments using a CAR expression vector showed that CAR could upregulate the CYP3A7 reporter and that this regulation was also dependent on the presence of the XREM containing region.

The fact that CAR is not activated in the same sense as PAR suggests that PAR is indeed the major “xenobiotic sensor” in the liver. This is supported by transient transfection experiments where the PAR and CAR expressing plasmids have been titrated (Goodwin et al., 1999). Consequently, substances that activates PAR, and thereby expression of CYP3A4 and CYP3A7, are likely to have an effect drug metabolism and steroid homeostasis.

According to the invention, the identification of (i) PAR as the transcription factor responsible for xenobiotic mediated regulation of CYP3A7, and (ii) upstream response elements for PAR in the CYP3A7 promoter, can be used for the development of screening methods for medically useful agents, e.g. agents modulating metabolism of endogenous and/or exogenous compounds, agents modulating drug interaction, and/or agents modulating toxicity or bioavailability of drugs. Such methods could also be used for screening environmental pollutants for potential endocrine disrupting capacity (cf. Masuyama et al., 2000).

Consequently, in a first aspect this invention provides an isolated human CYP3A7 promoter region comprising the dNR1 nucleotide sequence shown as positions 3425 to 3439 in SEQ ID NO: 1 or the dNR2 nucleotide sequence shown as positions 3469 to 3486 in SEQ ID NO: 1. Such a promoter region could more specifically comprise the XREM nucleotide sequence shown as positions 1832 to 3655 in SEQ ID NO: 1. Alternatively, the said isolated human CYP3A7 promoter region can comprise the dNR3 nucleotide sequence shown as positions 3855 to 3869 in SEQ ID NO: 1. The term “promoter region” refers to a region of DNA that functions to control the transcription of one or more genes, and is structurally identified by the presence of a binding site for DNA-dependent RNA polymerase and of other DNA sequences on the same molecule which interact to regulate promoter function.

An “isolated” nucleic acid is a nucleic acid molecule the structure of which is not identical to that of a naturally occurring nucleic acid or to that of any fragment of a naturally occurring genomic nucleic acid spanning more than one gene.

In another aspect, the invention provides a nucleic acid comprising a nucleotide sequence selected from the group consisting of nucleotides 3425 to 3439 of SEQ ID NO: 1, nucleotides 3469 to 3486 of SEQ ID NO:1, nucleotides 3855 to 3869 of SEQ ID NO:1, and nucleotides 3330 to 3551 of SEQ ID NO:1, operably linked to a heterologous coding sequence. A “heterologous coding sequence” is any coding sequence other than one that naturally encodes a CYP3A7 protein.

In another aspect, the invention provides a recombinant construct comprising the human CYP3A7 promoter region as defined above. In the said recombinant construct, the human CYP3A7 promoter region can be operably linked to a gene encoding a detectable product, e.g. a human CYP3A7 gene essentially having a nucleic acid sequence set forth as SEQ ID NO: 2.

The term “linked” indicates that a nucleotide sequence encoding a gene product and an CYP3A7 promoter, or an active fragment thereof, are located within a continuous nucleic acid sequence. The term “operably linked” means that a nucleotide sequence, which can encode a gene product, is linked to the CYP3A7 promoter such that the CYP3A7 promoter regulates expression of the gene product under appropriate conditions. Two nucleotide sequences that are operably linked contain elements essential for transcription, including, for example, a TATA box.

The recombinant construct according to the invention could comprise a reporter gene. As used herein, the term “reporter gene” means a gene encoding a gene product that can be identified using simple, inexpensive methods or reagents and that can be operably linked to a CYP3A7 promoter or an active fragment thereof. Reporter genes such as, for example, a luciferase, β-galactosidase, alkaline phosphatase, or green fluorescent protein reporter gene, can be used to determine transcriptional activity in screening assays according to the invention (see, for example, Goeddel (ed.), Methods Enzymol., Vol. 185, San Diego: Academic Press, Inc. (1990); see also Sambrook, supra).

In another aspect the invention provides a vector comprising the recombinant construct as defined above. The term “vector” refers to any carrier of exogenous DNA that is useful for transferring the DNA to a host cell for replication and/or appropriate expression of the exogenous DNA by the host cell. A host cell stably transformed with the recombinant construct is an additional aspect of the invention. Such a host cell can be a prokaryotic cell, a unicellular eukaryotic cell, or a cell derived from a multicellular organism. The methods employed to effect introduction of the vector into the host cell are standard methods well known to a person familiar with recombinant DNA methods. The term “transformed” or “transfected” refers to the process by which exogenous DNA is transferred into an appropriate host cell.

In a further important aspect, this invention is useful in screening for pharmacological agents that modulate CYP3A7 levels by affecting the transcription of the CYP3A7 gene. As used herein, the term “agent” means a biological or chemical compound such as a simple or complex organic molecule, a peptide, a protein or an oligonucleotide.

For screening purposes, appropriate host cells can be transformed with a vector having a reporter gene under the control of the human CYP3A7 promoter according to this invention. The expression of the reporter gene can be measured in the presence or absence of an agent with known activity (i.e. a standard agent) or putative activity (i.e. a “test agent” or “candidate agent”). A change in the level of expression of the reporter gene in the presence of the test agent is compared with that effected by the standard agent. In this way, active agents are identified and their relative potency in this assay determined.

Consequently, this invention includes a method for the identification of an agent capable of modulating CYP3A7 promoter activity, said method comprising the steps

(i) contacting a candidate agent with the human CYP3A7 promoter according to the invention; and

(ii) determining whether said candidate agent modulates expression of the CYP3A7 gene.

More specifically, such a method could comprise the steps:

(i) providing a cell comprising the CYP3A7 promoter region, operably linked to a reporter gene;

(ii) determining a first level of expression of the said reporter gene;

(iii) contacting the said cell with a candidate agent; and

(iv) determining a second level of expression of the reporter gene in the presence of the candidate agent, wherein a difference between the first and second levels of expression indicates that the candidate agent modulates CYP3A7 promoter activity.

It will be understood that agents acting on the human CYP3A7 promoter can be identified by, as an additional step, analyzing direct binding interactions between the candidate agent and the human CYP3A7 promoter. Interactions with large molecules may be studied using techniques such as gel shift analysis, footprinting or NMR (see Latchman, D.S. (Ed.) (1995) Methods for studying transcription factors. In: Eukaryotic transcription factors. Academic Press, London, pp. 17-44). Small molecule compounds which appear to bind reversibly to double stranded DNA without intercalation between DNA base pairs have been defined. Methods are described by which this non-intercalative binding can be characterized using ultraviolet spectrometry, fluorimetry with ethidium as a probe, viscometry and other hydrodynamic techniques, circular dichroism and nuclear magnetic resonance spectrometry (See Baguley, B. C. (1982) Nonintercalative DNA-binding antitumor compounds. Mol Cell Biochem 43: 167-181; Gmeiner, W. H. (1998) NMR spectroscopy as a tool to investigate the structural basis of anticancer drugs. Curr Med Chem 5(2):115-135; Wemmer, D. E. & Williams, P. G. (1994) Use of nuclear magnetic resonance in probing ligand-macromolecule interactions. Methods Enzymol. 239:739-767).

In another important aspect, this invention provides a method for identifying an agent which modulates the association of a CYP3A7 promoter and a transcription factor, comprising the steps:

(i) contacting a CYP3A7 promoter region according to the invention with said transcription factor;

(ii) determining a first association between the CYP3A7 promoter region and the transcription factor;

(iii) contacting the said CYP3A7 promoter region and said transcription factor with a candidate agent; and

(iv) determining a second association between the CYP3A7 promoter region and the transcription factor in the presence of the candidate agent, wherein a difference between the first and the second association indicates that the candidate agent modulates the association of the CYP3A7 promoter and the transcription factor.

The said method could involve e.g. a solid phase in vitro binding assay, or a cell based transcription assay, both of which are well known to the skilled person. When the method involves a cell-based assay, it could comprise the following steps:

(i) providing a cell comprising the CYP3A7 promoter region, operably linked to a reporter gene, and the said transcription factor;

(ii) determining a first level of expression of the said reporter gene;

(iii) contacting the said cell with a candidate agent; and

(iv) determining a second level of expression of the reporter gene in the presence of the candidate agent, wherein a difference between the first and second levels of expression indicates that the candidate agent modulates the association of the CYP3A7 promoter and the transcription factor.

In preferable forms of the invention, the said transcription factor could be the Pregnane Activated Receptor (PAR; Bertilsson et al., 1998) or the Constitutive Androstane Receptor (CAR) (GenBank™ accession number NM 005122).

### Materials and Methods

Isolation of Genomic Clones

Two BAC clones, BAC 97c20 and BAC 156P02, were identified by hybridization screening to contain the CYP3A4 promoter. The screening was done by using a nucleotide fragment (corresponding to positions 9364 to 10434 in GenBank™ Accession No. AF185589) specific for the CYP3A4 promoter. The BAC clones were cleaved with BamHI and the obtained fragments were ligated into pBluescript II KS(+) (Stratagene). The obtained clones were screened by PCR for the presence of the CYP3A4 promoter by using primers specific for the published CYP3A4 promoter (GenBank™ Accession No. AF185589). The sequence for the

| | |
|---|---|
| 5′primer was | |
| 5′-TTAATGACCTAAGAAGTCACCAG-3′ | (SEQ ID NO: 5), |
| and for the 3′primer: | |
| 5′-CGCCTCTCTCTTGCCCTTGTC-3′ | (SEQ ID NO: 6). |

One clone containing 11.4 kb was sequenced and shown to contain the CYP3A4 promoter sequence. Screening with CYP3A7 specific primers,

| | |
|---|---|
| 5′-GACCTAAGAAGATGGAGTGG-3′ | (SEQ ID NO: 7), and |
| 5′-CATAAAATCTATTAACTCTCCT-3′ | (SEQ ID NO: 8), |

did not identify any clones containing the CYP3A7 promoter.

After identification of the XREM as the PAR responsive region new XREM specific primers were designed and used to rescreen the original clones. The sequences of the primers were

| | |
|---|---|
| 5′-TCTAGAGAGATGGTTCATTCCTTTCA-3′ | (SEQ ID NO: 9) and |
| 5′-TGTTCTTGTCAGAAGTTCAGCTT-3′ | (SEQ ID NO: 10). |

A clone that contained part of the CYP3A7 promoter was isolated and sequenced. This clone contained the CYP3A7 promoter between position −11133 and −3188. In order to isolate the reminding part of the promoter PCR was performed on genomic DNA. The sequences of the primers were

| | |
|---|---|
| 5′-GCTGGGCATGGTGGTATACCTGTAGTA-3′ | (SEQ ID NO: 11) and |
| 5′-agcggatccTGCTGCTGTTTGCTGGGCTGTGT-3′ | (SEQ ID NO: 12). |

The latter primer contains a BamHI site and additional 3 nucleotides not present in genomic DNA (indicated by lowercase letters). This was done in order to clone the CYP3A7 promoter sequence into a reporter plasmid, see below. The PCR fragment was cloned into pCR2.1/TOPO (Invitrogen) to create the plasmid CYP3A7 −3604/+53-Topo. The fragment obtained by PCR was sequenced. Several independent gene walking experiments confirmed the sequence.

Plasmid Contructs

Manipulations of DNA were done using enzymes and the Rapid DNA Ligation Kit supplied by Roche. The plasmids were transformed into TOP10 cells (Invitrogen) and grown in LB. Plasmids was isolated using the Plasmid Maxi kit (Qiagen). The CYP3A4 promoter was amplified by PCR, using the primers

| | |
|---|---|
| 5′-CAGCACTGAACTCTAGCCTGGGCAACA-3′ | (SEQ ID NO: 13), and |
| 5′-agcggatccTGCTGCTGTTTGCTGGGCTGTGT-3′ | (SEQ ID NO: 12), |

in order to introduce a BamHI site immediately downstream of position +53. (Lower case letters denotes bases not present in the CYP3A7 sequence.)

A BamHI/KpnI fragment from the PCR product was ligated into the BglII and KpnI sites of the pGL3-Basic Vector (Promega) in order to create the vector CYP3A4-3192. A BamHI/EcoRV encompassing −10466 and −3096 of the CYP3A4 promoter sequence was cloned into the pCR2.1/TOPO vector (Invitrogen). A KpnI fragment from the obtained vector were cloned into the KpnI site of the CYP3A4-3192 vector. The final vector, CYP3A4-10466, contained CYP3A4 promoter sequence from −10466 and +53 cloned into the KpnI and the BglII sites, the latter was inactivated upon cloning, of the pGL3-Basic Vector. The vector also contained a SacI site just upstream of the promoter sequence, this site originated from the pCR2.1/TOPO vector.

A BamHI/HindIII fragment from CYP3A7-3604/+53-Topo was cloned into pBleuscript II KS(+) in order to create the plasmid pBS-CYP3A7-3604. The XhoI/HindIII fragment encompassing −7803 and −3604 of the CYP3A7 promoter was cloned into pBS-CYP3A7-3604 to create pBS CYP3A7-7803. A BamHI/SmaI fragment from this plasmid was cloned into pGL3-Basic in order to create the plasmid 3A7-6444. The 3A7-9302 plasmid was created by ligating a NheI/SmaI fragment (position −9302/−6444) from the CYP3A7 promoter into the 3A7 -6444 plasmid. The 3A7-7478 plasmid was created by cutting the CYP3A7-9302 with SpeI and NheI and religating. The 3A7-6444 (−9302/−7479 ins) plasmid was created by ligating the -9302/-7479 NheI/SpeI fragment of the Cyp3A7 promoter into the NheI site of 3A7-6444.

Expression vector for full-length nuclear receptor PAR-2 and the pRSV-AF reporter plasmid have been described previously (Bertilsson et al., 1998). The CAR expression vector was prepared by amplifying full-length CAR by RT-PCR from total RNA prepared from C3A/HepG2 cells using the primers

| | |
|---|---|
| 5′-CGGAATTCTCAGCTGCAGATCTCCTGGA-3′ | (SEQ ID |
| | NO: 14), and |
| 5′-CGGGATCCATGGCCAGTAGGGAAGATGA-3′ | (SEQ ID |
| | NO: 15). |

hCAR was cloned into pcDNA-3 vector (Invitrogen) using restriction enzymes BamHI and EcoRI.

Cell Based Reporter Assays

Six well plates were seeded with 0.5×10⁶ C3A cells. The cells were maintained in MEM with phenol red containing L-glutamine and supplemented with nonessential amino acids. The medium also contained 10% foetal bovine serum (life Technologies). The cells were allowed to settle for 24 hours before the medium was replaced by medium containing 10% charcoal treated foetal bovine serum (Hyclone Laboratories). Each well was transfected with 0.1 μg RSV-AF, 2 μg of the indicated reporter and 0.5 μg of the expression vector for PAR-2, CAR or the empty expression vector, pcDNA3. FuGENE 6™ Transfection Reagent (Roche) was used according to recommendations from the manufacturer. After 24 hours the medium was replaced by fresh medium containing 10% charcoal treated foetal bovine serum. The cells were induced by the addition of either DMSO (vehicle) or the indicated compound at a concentration of 10 μM. After induction for 48 hours the medium was analysed for the alkaline phosphatase activity and the luciferase activity was measured in the cell lysate as described (Bertilsson et al., 1998). DMSO, rifampicin and clotrimazole were purchased from Sigma-Aldrich.

Throughout this description the terms “standard protocols” and “standard procedures”, when used in the context of molecular biology techniques, are to be understood as protocols and procedures found in an ordinary laboratory manual such as: Current Protocols in Molecular Biology, editors F. Ausubel et al., John Wiley and Sons, Inc. 1994, or Sambrook, J., Fritsch, E. F. and Maniatis, T., Molecular Cloning: A laboratory manual, 2nd Ed., Cold Spring Harbor Laboratory Press, Cold Spring Harbor, N.Y. 1989.

### EXAMPLES

### Example 1

### Cloning of the CYP3A4 and CYP3A7 Promoter Sequences

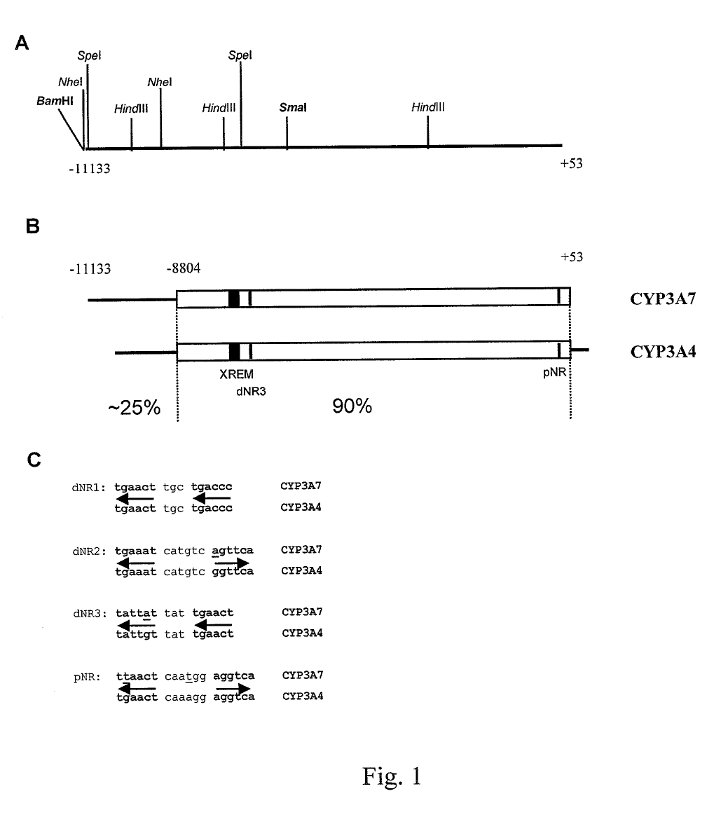

More than 10 kb of upstream promoter sequence for CYP3A4 and CYP3A7, respectively, was cloned and sequenced. Alignment of the two sequences indicates an extremely high sequence identity up to −8.8 kb (FIG. 1b). The sequence identity is 90%, which is the same degree of conservation as within the coding sequence. Above −8.8 kb the sequence identity is only random (25%). The published CYP3A4 promoter sequence (GenBank™ Accession No. AF185589) is identical sequence with the obtained CYP3A4 sequence, with exception for a few base pair changes, which could be due to polymorphism. None of these changes was located in any regions known to be important for the regulation of the CYP3A4 promoter. In similarity with Goodwin et al., we noted 5 bp mismatches compared to the previously published sequence for the CYP3A4 proximal promoter (Goodwin et al., 1999; Hashimoto et al., 1993).

The sequence of the obtained CYP3A7 promoter is shown as SEQ ID NO: 1. Transcription start is at position 11,134 in SEQ ID NO: 1. Positions 10,120 to 11,129 in SEQ ID NO: 1 correspond to positions 3 to 1012 in the previously published proximal CYP3A7 promoter (SEQ ID NO: 4).

### Example 2

### Identification of the PAR Responsive Element in the CYP3A7 Promoter

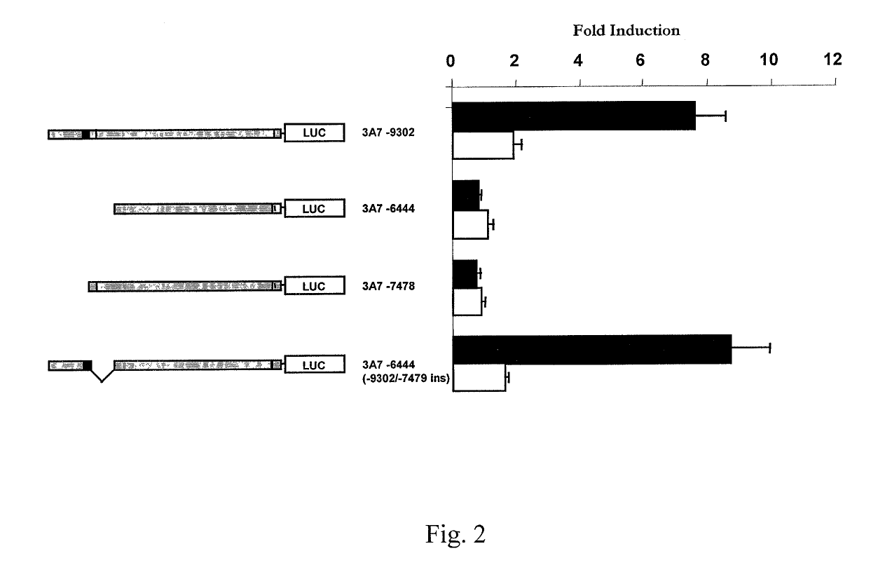

Transient transfection experiments using different deletion mutants of the CYP3A7 promoter identified the region between −9302 and −7479 (positions 1832 to 3655 in SEQ ID NO: 1) to contain the major PAR responsive element (FIG. 2). This region encompasses the XREM previously identified (Goodwin et al., 1999). The induction of the CYP3A7 reporter was dependent on exogenously added PAR (compare the white and the black bars in FIG. 2).

We conclude, based on sequence homology, that the CYP3A7 promoter contains a XREM sequence located between −7804 and −7583 (positions 3330 to 3551 in SEQ ID NO: 1). A closer examination of the distal nuclear receptor binding motifs (dNRs) previously identified in CYP3A4 (Goodwin at al., 1999) showed that dNR1 (positions 3425 to 3439 in SEQ ID NO: 1) was identical to the corresponding motif in CYP3A4 (FIG. 1c). The motifs dNR2 (positions 3469 to 3486 in SEQ ID NO: 1) and dNR3 (positions 3855 to 3869 in SEQ ID NO: 1) each contained a single base pair change in comparison with CYP3A4. There are two base pair changes in the CYP3A7 proximal PAR response element (pNR) compared with pNR in CYP3A4 (Pascussi et al., 1999).

### Example 3

### CAR Can Upregulate the CYP3A7 Promoter Via the XREM

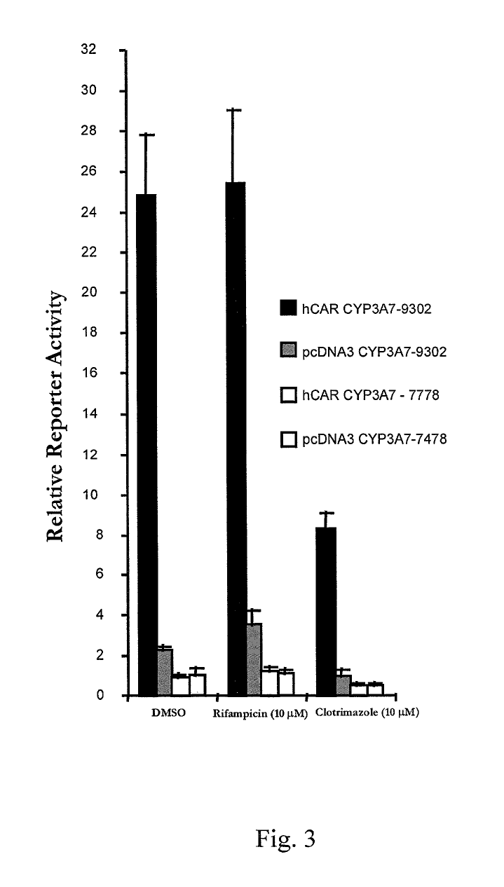

PAR shares DNA binding specificity and has overlapping ligand specificity with the nuclear receptor CAR (Moore et al., 2000). In order to investigate whether CAR could influence the activity of the CYP3A7 promoter we transfected cells with CYP3A7 reporters that either contained (CYP3A7-9302) or lacked (CYP3A7-7478) the region containing the XREM. The results (FIG. 3) indicate that CAR is a strong activator of CYP3A7 and that this is dependent on the presence of the XREM containing region. The CAR dependent induction of CYP3A7 reporter could be reduced by using a CAR inhibitor, clotrimazole.

### REFERENCES

Barwick, J. L., Quattrochi, L. C., Mills, A. S., Potenza, C., Tukey, R. H., and Guzelian, P. S. (1996). Trans-species gene transfer for analysis of glucocorticoid-inducible transcriptional activation of transiently expressed human CYP3A4 and rabbit CYP3A6 in primary cultures of adult rat and rabbit hepatocytes. Molecular Pharmacology 50, 10-6.

Bertilsson, G., Heidrich, J., Svensson, K., Asman, M., Jendeberg, L., Sydow-Backman, M., Ohlsson, R., Postlind, H., Blomquist, P., and Berkenstam, A. (1998). Identification of a human nuclear receptor defines a new signaling pathway for CYP3A induction. Proceedings of the National Academy of Sciences of the United States of America 95, 12208-13.

Blumberg, B., Sabbagh, W., Jr., Juguilon, H., Bolado, J., Jr., van Meter, C. M., Ong, E. S., and Evans, R. M. (1998). SXR, a novel steroid and xenobiotic-sensing nuclear receptor. Genes & Development 12, 3195-205.

Goodwin, B., Hodgson, E., and Liddle, C. (1999). The orphan human pregnane X receptor mediates the transcriptional activation of CYP3A4 by rifampicin through a distal enhancer module. Molecular Pharmacology 56, 1329-39.

Hakkola, J., Raunio, H., Purkunen, R., Pelkonen, O., Saarikoski, S., Cresteil, T., and Pasanen, M. (1996). Detection of cytochrome P450 gene expression in human placenta in first trimester of pregnancy. Biochemical Pharmacology 52, 379-83.

Hakkola, J., Pelkonen, O., Pasanen, M., and Raunio, H. (1998). Xenobiotic-metabolizing cytochrome P450 enzymes in the human feto-placental unit: role in intrauterine toxicity. Critical Reviews in Toxicology 28, 35-72.

Hashimoto, H., Toide, K., Kitamura, R., Fujita, M., Tagawa, S., Itoh, S., and Kamataki, T. (1993). Gene structure of CYP3A4, an adult-specific form of cytochrome P450 in human livers, and its transcriptional control. European Journal of Biochemistry 218, 585-95.

Jones, S. A., Moore, L. B., Shenk, J. L., Wisely, G. B., Hamilton, G. A., McKee, D. D., Tomkinson, N. C. O., LeCluyse, E. L., Lambert, M. H., Willson, T. M., Kliewer, S. A., and Moore, J. T. (2000). The pregnane x receptor: A promiscuous xenobiotic receptor that has diverged during evolution. Molecular Endocrinology 14, 27-39.

Kivisto, K. T., Griese, E. U., Fritz, P., Linder, A., Hakkola, J., Raunio, H., Beaune, P., and Kroemer, H. K. (1996). Expression of cytochrome P 450 3A enzymes in human lung: a combined RT-PCR and immunohistochemical analysis of normal tissue and lung tumours. Naunyn-Schmiedebergs Archives of Pharmacology 353, 207-12.

Kliewer, S. A., Moore, J. T., Wade, L., Staudinger, J. L., Watson, M. A., Jones, S. A., McKee, D. D., Oliver, B. B., Willson, T. M., Zetterstrom, R. H., Perlmann, T., and Lehmann, J. M. (1998). An orphan nuclear receptor activated by pregnanes defines a novel steroid signaling pathway. Cell 92, 73-82.

Kocarek, T. A., Schuetz, E. G., Strom, S. C., Fisher, R. A., and Guzelian, P. S. (1995). Comparative analysis of cytochrome P4503A induction in primary cultures of rat, rabbit, and human hepatocytes. Drug Metabolism & Disposition 23, 415-21.

Kolars, J. C., Lown, K. S., Schmiedlin-Ren, P., Ghosh, M., Fang, C., Wrighton, S. A., Merion, R. M., and Watkins, P. B. (1994). CYP3A gene expression in human gut epithelium. Pharmacogenetics 4, 247-59.

Lehmann, J. M., McKee, D. D., Watson, M. A., Willson, T. M., Moore, J. T., and Kliewer, S. A. (1998). The human orphan nuclear receptor PXR is activated by compounds that regulate CYP3A4 gene expression and cause drug interactions. Journal of Clinical Investigation 102, 1016-23.

Masuyama, H., Hiramatsu, Y., Kunitomi, M., Kudo, T., and MacDonald, P. N. (2000). Endocrine disrupting chemicals, phthalic acid and nonylphenol, activate Pregnane X receptor-mediated transcription. Molecular Endocrinology 14, 421-428.

Moore, L. B., Parks, D. J., Jones, S. A., Bledsoe, R. K., Consler, T. G., Stimmel, J. B., Goodwin, B., Liddle, C., Blanchard, S. G., Willson, T. M., Collins, J. L., and Kliewer, S. A. (2000). Orphan nuclear receptors constitutive androstane receptor and pregnane X receptor share xenobiotic and steroid ligands. Journal of Biological Chemistry 275, 15122-7.

Murray, G. I., McFadyen, M. C., Mitchell, R. T., Cheung, Y. L., Kerr, A. C., and Melvin, W. T. (1999). Cytochrome P450 CYP3A in human renal cell cancer. British Journal of Cancer 79, 1836-42.

Nelson, D. R. et al. (1996). P450 superfamily. Update on new sequences, gene mapping, accession numbers and nomenclature. Pharmacogenetics 6, 1-42 Pascussi, J. M., Jounaidi, Y., Drocourt, L., Domergue, J., Balabaud, C., Maurel, P., and Vilarem, M. J. (1999). Evidence for the presence of a functional pregnane X receptor response element in the CYP3A7 promoter gene. Biochemical & Biophysical Research Communications 260, 377-81.

Shimada, T., Yamazaki, H., Mimura, M., Wakamiya, N., Ueng, Y. F., Guengerich, F. P., and Inui, Y. (1996). Characterization of microsomal cytochrome P450 enzymes involved in the oxidation of xenobiotic chemicals in human fetal liver and adult lungs. Drug Metabolism & Disposition 24, 515-22.

Schuetz, J. D., Kauma, S., and Guzelian, P. S. (1993). Identification of the fetal liver cytochrome CYP3A7 in human endometrium and placenta. Journal of Clinical Investigation 92, 1018-24.

Sueyoshi, T., Kawamoto, T., Zelko, I., Honkakoski, P., and Negishi, M. (1999). The repressed nuclear receptor CAR responds to phenobarbital in activating the human CYP2B6 gene. Journal of Biological Chemistry 274, 6043-6.

Tateishi, T., Nakura, H., Asoh, M., Watanabe, M., Tanaka, M., Kumai, T., Takashima, S., Imaoka, S., Funae, Y., Yabusaki, Y., Kamataki, T., and Kobayashi, S. (1997). A comparison of hepatic cytochrome P450 protein expression between infancy and postinfancy. Life Sciences 61, 2567-74.

Xie, W., Barwick, J. L., Downes, M., Blumberg, B., Simon, C. M., Nelson, M. C., Neuschwander-Tetri, B. A., Bruntk, E. M., Guzelian, P. S., and Evans, R. M. (2000). Humanized xenobiotic response in mice expressing nuclear receptor SXR. Nature 406, 435-439.

Yang, H. Y., Lee, Q. P., Rettie, A. E., and Juchau, M. R. (1994). Functional cytochrome P4503A isoforms in human embryonic tissues: expression during organogenesis. Molecular Pharmacology 46, 922-8.

What is claimed is:

1. An isolated human CYP3A7 promoter region comprising the dNR1 nucleotide sequence shown as positions 3425 to 3439 in SEQ ID NO: 1.

2. An isolated human CYP3A7 promoter region comprising the dNR2 nucleotide sequence shown as positions 3469 to 3486 in SEQ ID NO: 1.

3. The isolated human CYP3A7 promoter region of claim 1, comprising the XREM nucleotide sequence shown as positions 1832 to 3655 in SEQ ID NO: 1.

4. An isolated human CYP3A7 promoter region comprising the dNR3 nucleotide sequence shown as positions 3855 to 3869 in SEQ ID NO: 1.

5. The isolated human CYP3A7 promoter region of claim 1, comprising the nucleotide sequence set forth as SEQ ID NO: 1.

6. A recombinant construct comprising the human CYP3A7 promoter region of claim 1.

7. The recombinant construct of claim 6, wherein the human CYP3A7 promoter region is operably linked to a nucleic acid molecule comprising a nucleotide sequence that encodes a detectable product.

8. The recombinant construct of claim 7, wherein the nucleic acid molecule comprises a sequence set forth as SEQ ID NO:2.

9. A vector comprising the recombinant construct of claim 6.

10. A host cell stably transformed with the vector of claim 9.

11. A method for identification of an agent capable of modulating CYP3A7 promoter activity, the method comprising:
(i) contacting a candidate agent with the human CYP3A7 promoter region of claim 1; and
(ii) determining whether the candidate agent modulates CYP3A7 promoter activity.

12. A method for identification of an agent capable of modulating CYP3A7 promoter activity, the method comprising:
(i) providing a cell comprising the human CYP3A7 promoter region of claim 1, operably linked to a reporter gene;
(ii) determining a first level of expression of the reporter gene;
(iii) contacting the cell with a candidate agent; and
(iv) determining a second level of expression of the reporter gene in the presence of the candidate agent, wherein a difference between the first and second levels of expression indicates that the candidate agent modulates CYP3A7 promoter activity.

13. A method for identifying an agent that modulates the association of a CYP3A7 promoter and a transcription factor, the method comprising:
(i) contacting the CYP3A7 promoter region of claim 1 with a transcription factor;
(ii) determining a first association between the CYP3A7 promoter region and the transcription factor;
(iii) contacting the CYP3A7 promoter region and the transcription factor with a candidate agent; and
(iv) determining a second association between the CYP3A7 promoter region and the transcription factor in the presence of the candidate agent, wherein a difference between the first and the second association indicates that the candidate agent modulates the association of the CYP3A7 promoter and the transcription factor.

14. The method of claim 13, wherein the method is a solid phase in vitro binding assay.

15. The method of claim 13, wherein the method is a cell based transcription assay.

16. A method for identifying an agent that modulates the association of a CYP3A7 promoter and a transcription factor, the method comprising:
(i) providing a cell comprising the CYP3A7 promoter region of claim 1, operably linked to a reporter gene, and the transcription factor;
(ii) determining a first level of expression of the reporter gene;
(iii) contacting the cell with a candidate agent; and
(iv) determining a second level of expression of the reporter gene in the presence of the candidate agent, wherein a difference between the first and second levels of expression indicates that the candidate agent modulates the association of the CYP3A7 promoter and the transcription factor.

17. The method according to claim 13, wherein the transcription factor is the Pregnane Activated Receptor (PAR).

18. The method according to claim 13, wherein the transcription factor is the Constitutive Androstane Receptor (CAR).

19. The isolated human CYP3A7 promoter region of claim 1, comprising the XREM nucleotide sequence shown as positions 3330 to 3551 in SEQ ID NO:1.

20. The isolated human CYP3A7 promoter region of claim 1, further comprising the dNR2 nucleotide sequence shown as positions 3469 to 3486 in SEQ ID NO: 1.

21. The isolated human CYP3A7 promoter region of claim 20, further comprising the dNR3 nucleotide sequence shown as positions 3855 to 3869 in SEQ ID NO: 1.

22. A nucleic acid comprising a nucleotide sequence selected from the group consisting of nucleotides 3425 to 3439 of SEQ ID NO:1, nucleotides 3469 to 3486 of SEQ ID NO:1, nucleotides 3855 to 3869 of SEQ ID NO:1, and nucleotides 3330 to 3551 of SEQ ID NO: 1, operably linked to a heterologous coding sequence.
